(19) United States
(12) Patent Application Publication
(10) Pub. No.: US 2008/0171968 A1
(43) Pub. Date: Jul. 17, 2008

(54) METHODS OF ADMINISTERING INJECTABLES TO A JOINT WITH A NEEDLE-FREE INJECTION SYSTEM

(75) Inventors: Richard Stout, West Linn, OR (US); Nicolas Duval, Montreal (CA)

Correspondence Address: KOLISCH HARTWELL, P.C., 520 SW YAMHILL STREET, Suite 200, PORTLAND, OR, 97204, US

(73) Assignee: BIOJECT, INC., Tualatin, OR (US)

(21) Appl. No.: 11/682,452

(22) Filed: Mar. 6, 2007

Related U.S. Application Data
(60) Provisional application No. 60/884,990, filed on Jan. 15, 2007.

(51) Int. Cl. A61M 5/30

(52) U.S. Cl. 604/71

(57) ABSTRACT

A method of delivering an injectable to a joint region with a needle-free injection system having a chamber with a first end and a second end, an orifice of a pre-selected size in the first end, and a plunger mechanism for ejecting fluids from within the chamber, through the orifice, and onto a target site. When the chamber is filled with an injectable, the plunger mechanism is configured to eject a selected amount of the injectable onto the target site at a pre-determined rate. A skin site adjacent a joint is selected. The orifice is placed in an injection position relative to the skin site, whereby the skin site is the target site. The injectable is ejected onto the skin site with the plunger mechanism, whereby at least a portion of the injectable is delivered through the skin site to intradermal, subcutaneous, intramuscular, and/or intra-articular regions of the joint.

### CROSS-REFERENCE TO RELATED APPLICATION

This application claims priority under 35 U.S.C. § 119(e) to U.S. Provisional Patent Application Ser. No. 60/884,990 entitled “Method of Administering Medicaments to a Joint with a Needle-Free Injection System,” filed Jan. 15, 2007, the complete disclosure of which is herein incorporated by reference for all purposes.

### BACKGROUND

Doctors treat a wide variety of injury to, and disease of, the joints, such as the hip, knee, ankle, toes, shoulder, elbow, wrist, fingers, or vertebrae. Some of the most frequent conditions treated by doctors include: (a) diseased or inflamed connective tissue (e.g. arthritis, bursitis, tendonitis, etc.); (b) acute and chronic injury to the bones or connective tissue in a joint, (e.g. torn cartilage and/or ligaments, recurrent dislocations, carpal tunnel syndrome, herniated disks, etc.) and (c) loose bodies of bone and/or cartilage in a joint. For example, surgeons may perform either open surgery or arthroscopic surgery to treat these conditions. Doctors may also treat these conditions by injecting various injectables into the joint, such as antibiotics, steroids, analgesics, medications, saline solution, and/or other fluids.

Open joint surgery is an invasive type of surgery that requires a doctor (e.g. an orthopedic surgeon) to make one or more incisions in the skin and tissues of the injured joint in order to obtain full access to the joint. The incision must be large enough for the surgeon to view and repair the damaged and/or diseased tissue. Depending on the extent of the injury and the size of the incision, the surgeon may use either general or local anesthesia, and the patient may be treated on an in-patient or out-patient basis.

Arthroscopic surgery is a less invasive type of surgical procedure that utilizes an arthroscope to visualize, diagnose, and/or treat a joint injury. The word arthroscopy comes from two Greek words, “arthro” (joint) and “skopein” (to look). The term literally means “to look within the joint.” In an arthroscopic examination, a surgeon makes a small incision in the patient's skin (about the size of a buttonhole) for insertion of the arthroscope, which is a pencil-sized tube having one or more lenses, and a lighting system to magnify and illuminate the structures inside the joint. Light captured by the lens is transmitted through fiber optics to an external camera, which records the image, and enables the surgeon to see the interior of the joint on a display screen. The television camera attached to the arthroscope displays the image of the joint on a television screen, allowing the surgeon to look throughout the joint at the cartilage, ligaments, and bones. The surgeon can determine the amount or type of injury, and then repair or correct the problem, if it is necessary. Specifically, the surgeon makes one or more other small incisions for insertion of the surgical instruments used to treat the joint.

Arthroscopic surgery, although much easier in terms of recovery than open surgery, still requires the use of anesthetics and special equipment in a hospital operating room or outpatient surgical suite. Arthroscopic surgery may be used for many types of joint surgery, including but not limited to: (a) treatment of rotator cuff injuries; (b) repair or resection of torn cartilage (e.g. the meniscus) from knees or shoulders; (c) reconstruction of the ACL or MCL in the knee; (d) removal of inflamed lining (synovium) from knees, shoulders, elbows, wrists, or ankles; (e) release of carpal tunnel; (f) repair of torn ligaments; and (g) removal of loose bone or cartilage from knees, shoulders, elbows, ankles, and wrists. Depending on the joint or the suspected injury, patients may be given either a general or a local anesthetic. However, due to the risks associated with the use of general anesthesia, and the amount of recovery time required for patients receiving general anesthesia, more and more surgeons are choosing local anesthesia for simple arthroscopic surgeries including biopsies, meniscectomies, removal of intra-articular foreign bodies, synovectomies, and intra-articular debridement for osteoarthritis.

In addition to open and arthroscopic joint surgery, other types of joint treatment have emerged as alternatives to treat conditions such as arthritis. For example, a steroid such as cortisone may be injected into the joint to alleviate pain, or a viscous gel-like substance may be injected to aid in lubrication of the joint as an alternative to major surgery on the arthritic joint.

For both open and arthroscopic joint surgery, as well as other types of joint treatment, administration of injectables (such as anesthetics, antibiotics, steroids, analgesics, medications, gels, saline solution, and/or other drugs and liquid injectables) has historically required the use of a needle and syringe. For example, a needle and syringe provides the standard mechanism for injecting an anesthetic intradermally, subcutaneously, and/or intra-articularly, prior to and/or during surgery. Injections using a needle and syringe may be painful, may cause anxiety for the patient, and may inadequately and/or inefficiently disperse the injectable within the target tissue. For the foregoing reasons there is a need for a method of administrating injectables to a joint without the use of the traditional needle and syringe.

### SUMMARY

The present disclosure provides a method of administering one or more injectables to a joint with a needle-free injection system. Use of a needle-free injection system may be less painful to the patient and may not invoke the typical fearful response in a patient when compared to the traditional needle and syringe method. Additionally, the needle-free injection system may administer the injectable more efficiently to the desired site, thereby reducing the amount of injectable required. A reduction in the amount of required injectable may in turn reduce the cost of a procedure, and/or minimize the risk of toxicity to the patient.

An exemplary method of administering an injectable to a joint using a needle-free injection system may include the step of selecting a skin site adjacent a joint and placing the needle-free injection system in an injection position relative to the skin site. A pre-selected amount of the injectable may then be delivered through the skin site. Whether an additional administration of the injectable is desired may then be determined. The joint procedure may thereafter proceed as desired.

### BRIEF DESCRIPTION OF THE DRAWINGS

FIG. 1 depicts a flowchart of an exemplary method of administration of an injectable to a joint with a needle-free injection system.

### DETAILED DESCRIPTION

In the latter part of the 1800's the first needle-free injection system (i.e. jet injection system) was invented because of the observation that fine leaks of diesel oil in high pressure lines would cause accidental injection into worker's hands with little pain. Needle-free injectors were marketed in the 1940's primarily for mass inoculations when vaccines became commercially available and needle/syringes were considered durable (reusable) devices. Needle-free injectors have been used to inject a wide variety of medications. Needle-free injectors typically include: (a) a chamber for holding an injectable liquid; (b) an orifice for directing pressurized injectable out of the injectable chamber and onto a target region; and (c) a plunger mechanism for ejecting a selectable amount of fluid from within the chamber, through the orifice, and onto a target site spaced at a pre-determined interval from the orifice. Examples of needle-free injectors can be found in U.S. Pat. Nos. 4,596,556; 4,790,824; 4,940,460; 4,941,880; 4,966,581; 5,064,413; 5,312,335; 5,312,577; 5,383,851; 5,399,163; 5,466,220; 5,503,627; 5,505,697; 5,520,639; 5,649,912; 5,746,714; 5,782,802; 5,893,397; 5,993,412; 6,096,002; 6,132,395; 6,264,629; 6,319,224; 6,383,168; 6,471,669; 6,506,177; 6,572,581; 6,585,685; 6,607,510; 6,641,554; 6,645,170; 6,648,850; 6,676,630; 6,689,093; 6,752,780; 6,752,781; 6,783,509; 6,883,222; 6,935,384; 7,131,961; and 7,156,823, the disclosures of which are herein incorporated by reference in their entirety for all purposes.

The chamber may include any chamber for receiving or storing a liquid injectable, and adapted to withstand high pressure. For example, the chamber may be a cylindrical chamber adapted to withstand pressures ranging from 4,000 pounds per square inch (psi) to 16,000 psi. The chamber may include first and second ends, and may come in various sizes to accommodate holding varying volumes of an injectable.

The orifice may include any orifice for directing pressurized injectable out of the injectable chamber and onto a target region (such as a patient's skin, joint, or other tissue). For example, the orifice may be positioned on an end of the chamber, and may have any pre-selected size, where the orifice size (as well as the viscosity of the injectable) affects the degree to which an injectable is pressurized upon application of force by the plunger mechanism. The degree to which the plunger mechanism pressurizes the injectable in turn affects the rate at which the injectable is ejected through the orifice. For example, the orifice may be substantially circular, and may have a diameter that ranges in size from 0.003 to 0.015 inches. The orifice may be selectively attachable, so as to permit a user to change the orifice size base on the desired application.

The plunger mechanism may be configured to eject a selectable amount of fluid from within the chamber, through the orifice, and onto a target site spaced at a pre-determined interval from the orifice. The plunger mechanism may include a plunger having a sliding seal that articulates within the chamber, and a drive mechanism for pushing the plunger.

Any drive mechanism may be used to push the plunger, including but not limited to (a) an electromagnet powered by electricity; (b) a hydraulic mechanism powered by pressurized liquid; (c) a pneumatic mechanism powered by compressed air, and/or (d) a compression spring. The plunger may also be manually actuated by a user. For example, compressed carbon dioxide, either from a tank connected with a high pressure gas line or from cartridges inside the device, may be used to pneumatically power the plunger. The drive mechanism may be configured to push the plunger a selected distance at a selected rate, where (a) the selected distance affects the amount of injectable ejected through the orifice, and (b) the selected rate affects the pressure applied by the drive mechanism to the injectable, and thus the rate at which the injectable is ejected through the orifice.

A needle-free injection system may be used to administer any injectable fluid. These fluids may include various compounds, including drugs, steroids, gels, anesthetics, antibiotics, analgesics, medications, hyaluronic acid, salts, and/or any other compounds. These injectables may have varying viscosities, where the viscosity of the injectable affects the degree to which an injectable is pressurized upon application of force by the plunger mechanism due to sheer between the injectable and the orifice. The degree to which the plunger mechanism pressurizes the injectable in turn affects the rate at which the injectable is ejected through the orifice.

A user may selectively adjust the rate of travel of an injectable as it is ejected onto a skin site. As discussed above, the rate of travel of the injectable at the moment it is ejected through the orifice is dependent on at least the following factors: (a) the size of the needle-free injection system's orifice; (b) the rate at which the drive mechanism pushes the plunger; and (c) the viscosity of the injectable. Once the injectable has been ejected through the orifice, it passes through the space between the orifice and the skin site. Because of air resistance, the distance of the orifice from the skin site affects the rate of the injectable at the moment it reaches the skin site. The distance of the orifice from the skin site also affects the area of skin that receives the injectable, as the injectable tends to spray outward as it is ejected though the orifice.

By adjusting the rate of travel of an injectable as it is ejected onto a skin site, and the area of the target skin site, a user may selectively adjust the dispersion pattern of an injectable through a target skin site. For example, a specific volume of injectable ejected onto a specific area of skin site at a relatively high rate generally penetrates deeper through the tissue beneath the skin site than the same volume of injectable ejected onto the same skin site at a lower rate. Further, a volume of injectable ejected at a specific rate onto a relatively small area of skin site, generally will be delivered in a more concentrated fashion to, and will penetrate deeper through, the tissue beneath the skin site than the same volume of injectable ejected at the same rate onto a larger target skin site.

Some needle-free injectors may include an attachable intra-dermal spacer, which may also be described as an adaptor. The adaptor may be used to ensure that the orifice is placed at a selected distance away from a target site. For example, the adaptor may include a proximal end selectively attachable to the end of a chamber adjacent the orifice, and a distal end adapted to engage a surface adjacent to a target site. When the distal end of the adaptor is engaged with a skin surface adjacent to the skin site, the adaptor may cause the orifice to be spaced at a selected interval from the skin site. The adaptor may also ensure that the orifice is aligned properly relative to the skin site. Various adaptors of various lengths may be provided so that a user may select the appropriate distance between the orifice and the target skin site.

The various components of a needle-free injector may either be intended for a single-use or may be reusable. For example, any component that comes in contact with bodily fluids may be disposable, whereas components that do not come into contact with bodily fluids may be either disposable or reusable provided they have been properly sanitized/sterilized.

Needle-free injectors provide for the ability to deliver medications intramuscularly, intradermally, subcutaneously and/or intra-articularly. For example, the Biojector® 2000 needle-free injector, which utilizes sterile, single-use syringes for individual injections, has been shown to effectively replace the needle and syringe for many injections.

As discussed above, the administration of injectables during the treatment of joints has historically required the use of a needle and syringe for injecting the injectable intradermally, subcutaneously, or intra-articularly. Because injections using a needle and syringe may be painful, may cause anxiety for the patient, and may inadequately disperse the injectable within the target tissue, it would be advantageous to use a needle-free injection system to administer injectables into any joint(s), such as the hip, knee, ankle, toes, shoulder, elbow, wrist, fingers, or vertebrae.

Additionally, because of the potential for more efficient dispersal of an injectable to the desired region, use of a needle-free injection system may require less injectable to achieve a desired result, thereby decreasing a patient's risk of receiving a toxic dose. For example, the same degree of local anesthesia for one portal in an arthroscopic surgical procedure on a knee may be achieved using 3.3 cc of xylocalne with a needle-free system method, instead of 10 cc of xylocalne as required with a traditional needle and syringe method.

A further advantage of administering an injectable with a needle-free injection system may include ease of use. For example, arthroscopic surgery of the knee, such as ACL reconstruction surgery, may be performed under local anesthesia. The main limitation to performing this intervention under local anesthesia is the difficulty in identifying the zones that need to be anesthetized over the anterolateral aspect of the thigh (corresponding to the proximal site of bone fixation of the synthetic ligament), and the anteromedial aspect of the leg (corresponding to the distal site of bone fixation of the synthetic ligament). Needle-free injection systems may be useful for overcoming this, as well as other limitations, associated with the use of a needle and syringe.

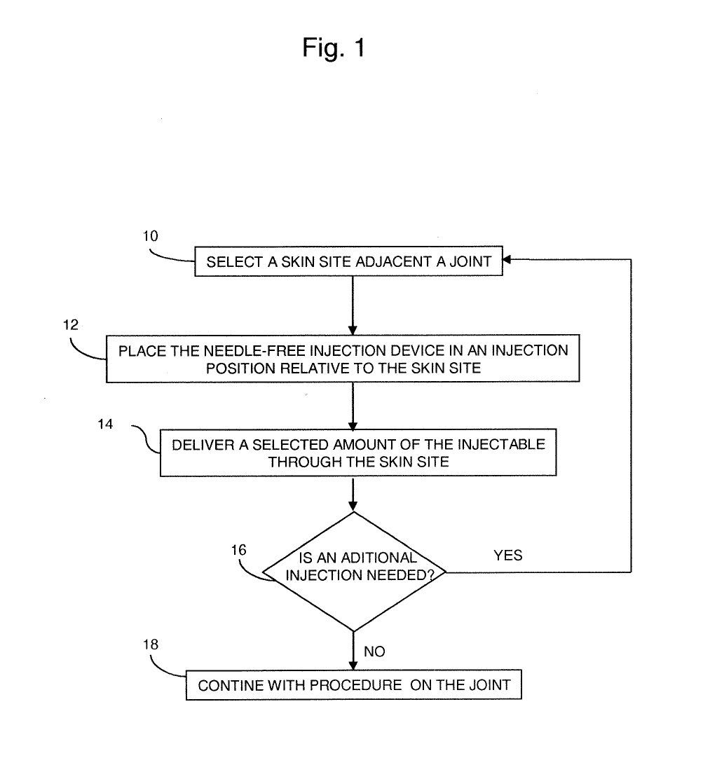

An exemplary method of administering an injectable to a joint using a needle-free system is illustrated in the flowchart shown in FIG. 1. A skin site adjacent a joint may be selected for administration of an injectable 10. A selected needle-free injection system may then be placed in a selected injection position relative to the skin site 12. For example, the selected needle-free injection system may include (a) a chamber that is at least partially filled with an injectable, (b) an orifice of a pre-selected size, and (c) a plunger mechanism configured to eject a selected amount of the injectable through the orifice and onto a target site spaced at a selected interval from the orifice at a pre-determined rate. The selected needle-free injection system may be positioned so that the orifice is aimed at the target skin site and is positioned at the selected interval. This interval may be ensured by using an adaptor, as discussed above.

The selected amount of the injectable may thereafter be delivered through the skin site 14. Specifically, the injectable may be ejected onto the skin site by actuating the plunger mechanism, whereby at least a portion of the injectable may be delivered through the skin site and into the intradermal, subcutaneous, intramuscular, and/or intra-articular regions of the joint. Whether an additional administration of the injectable is desired may thereafter be determined 16. Depending on whether additional injectable is desired, the joint procedure may then proceed as desired 18.

An additional exemplary method of delivering an injectable to a joint with a needle-free injection system may include a step of selecting a target joint of a subject, where the subject may be any vertebrate animal, including but not limited to human, providing a needle-free injection system having an injectable therein, and effecting a release of the injectable, whereby the injectable is accelerated to exit the needle-free injection system with sufficient energy to penetrate into a target tissue adjacent the target joint of the subject.

Studies have been performed to determine whether needle-free injection systems may be effectively used in place of a needle and syringe, to deliver injectables to a joint by documenting: (1) the subcutaneous and intramuscular dispersion pattern of bupivacaine injected into the anterolateral aspect of the distal thigh and to the anteromedial aspect of the proximal leg using a needle-free injection system; (2) the quality of the local anesthesia obtained when bupivacaine is injected to the anterolateral aspect of the distal thigh and to the anteromedial aspect of the proximal leg using a needle-free injection system; and (3) the difference in dispersion pattern and the quality of the anesthesia of two concentrations of bupivacaine (0.25% with epinephrine & 0.50% with epinephrine), administered over the anterolateral aspect of the distal thigh and the anteromedial aspect of the proximal leg using a need-free injection system.

As noted earlier the method of administering an injectable to a joint using a needle-free injection system may require less injectable to achieve a desired result than traditional needle and syringe method of injection. Thus the risk of toxicity to the patient is reduced. For example, bupivacaine is an anesthetic agent frequently used in surgical procedures performed under local anesthesia. Toxic side effects of this product are noted particularly at the neurological and cardiovascular levels. These toxic reactions are observed at serum bupivacaine levels in excess of 2mcg/ml. Other observed side effects included: dizziness, perioral numbness, slurred speech, delirium, convulsions, hypotension, bradycardia, ventricular arrythmis, and cardiac arrest.

An exemplary experimental study to determine the efficacy of administrating injectables (e.g. bupivacaine) with a needle-free injection system may be performed as follows:

The maximal allowable dose of bupivacaine may be determined in the following manner: 1 kg body weight=1 ml 0.25% bupivacaine with epinephrine. To date, no adverse toxic reactions have been noted following the intra-articular administration of bupivacaine. Each subject participating in this exemplary study may receive 18 ml of bupivacaine, a dose significantly smaller than what is used for knee surgeries performed under local anesthesia (40 ml). Therefore, no toxic side effects should be observed. The only anticipated risk would be an allergic reaction to the bupivacaine in an otherwise healthy individual without any known allergies.

An exemplary needle-free injection system to use may be the Biojector® 2000 needle-free system, which is a device that has been developed to deliver medications both intramuscularly and subcutaneously. However, depending on the desired application, any other needle-free injection system may be used to administer injectables to a joint. The Biojector® 2000 system typically includes three major components: the Biojector® (i.e. the drive mechanism), the CO₂ cartridge (i.e. the power source for the drive mechanism), and the sterile medication syringe (i.e. the chamber with a plunger and an attached orifice). A range of syringes may be provided that allow the Biojector® 2000 system to administer intradermal, subcutaneous, and/or intramuscular injections. For example, some syringes may be used to administer up to 1.0 ml of an injectable to a target skin site. The injectable is delivered under pressure by the compressed CO₂ cartridge, which is inserted into the Biojector®. When the Biojector's actuator is depressed, the CO₂ is released, causing the plunger to push the medication out of the syringe through the skin and into the underlying tissue.

Each subject of the exemplary study may have one lower extremity injected with 0.25% bupivacaine with epinephrine and the other leg injected with 0.5% bupivacaine with epinephrine. The procedure may be identical for each of the lower extremities and may proceed as follows:

First, joints associated with the lower extremities may be prepped for surgery by disinfecting the skin adjacent to the joint. After disinfecting the skin, six injections of a 1 ml solution of either 0.25% or 0.5% bupivacaine with epinephrine may be administered to the anterolateral aspect of the distal thigh using the needle-free injection system. The zone to be anesthetized may correspond to the proximal entry site of the artificial ligament used in ACL reconstruction. This region is lateral to the midline of the thigh 5 to 15 cm proximal to the upper end of the patella.

A second set of three injections of a 1 ml solution of 0.25% or 0.5% bupivacaine with epinephrine may be made to the anteromedial aspect of the proximal tibia. This region lies 2 to 10 cm distal to the distal end of the patella and corresponds to the distal exit site of the artificial ligament used in ACL reconstruction.

At 5, 15, 30, 60, 90, 120, 150 and 180 minutes post-injection, a sensory evaluation to light touch and pin prick may be performed on all subjects in order to assess the quality of the anesthesia. Once the zone of cutaneous anesthesia has been demarcated, the depth of the anesthesia may be assessed by introducing a 3 inch sterile needle into the soft tissues of the anesthetized region. Immediately following the assessment at 30 minutes, a Magnetic Resonance Image (MRI) of both knees may be obtained. The MRI may permit visualization of the bupivacaine dispersion pattern within the soft tissues surrounding the knee.

### EXAMPLES

The following qualitatively-described studies were performed to determine whether a needle-free injection system could be used to effectively anesthetize the skin of a patient proximal to a joint in order to prepare the patient for anesthesia of the intra-articular aspect of the joint using a needle and syringe. Specifically, the object was to determine whether use of a needle-free injection system for local anesthesia of the skin would (a) allow for the use of smaller quantities of anesthetic to provide the patient with effective local anesthesia; (b) provide more effective dispersion of anesthetic at the location of the injection; and (c) reduce a patient's fear of receiving the injection.

In these studies, adaptors were tested in conjunction with the needle-free injection system. As discussed above, an adaptor is a plastic cap, or spacer, of varying diameter and length that may be attached to the needle-free injection system adjacent to the injection orifice, and thereby enable a user to inject injectable from the orifice at a predetermined distance from the patients' skin. The objective in using the adaptor is to ensure a proper spacing of the orifice from the skin site so as to obtain more effective anesthesia of the intra-dermal layers of the skin.

The studies were performed on three patients with damaged knees that required arthroscopic surgery to repair the damage. A Biojector® 2000 needle-free injector was used to locally administer 0.3 cc of 1% xylocalne to each of the anterolateral aspect of the distal thigh, and the anteromedial aspect of the proximal leg. The quality of intra-dermal anesthesia was tested using (a) sensory evaluations to touch and pin prick; and (b) the dispersion pattern of the xylocalne by using an MRI to image the outer layers of the skin.

### Example 1

For the first patient, the needle-free injection system, with a 1.5 inch adaptor (i.e. spacer) and an orifice having a diameter of about 0.015 inches was used to locally administer 0.3 cc of 1% xylocalne with epinephrine to the intra-dermal layers of each of the anterolateral aspect of the distal thigh, and the anteromedial aspect of the proximal leg. Excellent intra-dermal anesthesia was achieved, after which a needle and syringe was used to anesthetize the lateral soft tissue of the knee with 10 cc of bupivacaine, and the intra-articular portion of the knee with 30 cc of bupivacaine. The arthroscopic surgery was then performed without any problems with the local anesthesia.

### Example 2

For the second patient, the needle-free injection system, with a 1.5 inch adaptor and an orifice having a diameter of about 0.015 inches was used to locally administer 0.3 cc of 1% xylocalne with epinephrine to the intra-dermal layers of each of the anterolateral aspect of the distal thigh, and the anteromedial aspect of the proximal leg. The needle-free injection system, with a 1.5 inch adaptor and an orifice having a diameter of about 0.045 inches was then used to locally administer 1 cc of 1% xylocalne with epinephrine to each of the anterolateral aspect of the thigh and the anteromedial aspect of the leg in an attempt to locally anesthetize the subcutaneous layers adjacent to the knee. Excellent anesthesia was observed, after which time a needle and syringe were used to anesthetize the lateral soft tissue of the knee with 10 cc of bupivacaine, and the intra-articular portion of the knee with 30 cc of bupivacaine. However, during the arthroscopic surgery, the patient experienced discomfort in the subcutaneous layers. A needle and syringe was then used to administer 10 cc 1% xylocalne to the subcutaneous tissue, and the surgery proceeded without any additional problems with the local anesthesia.

### Example 3

For the third patient, the needle-free injection system, with a 1.5 inch adaptor and an orifice having a diameter of about 0.0045 inches, was used to locally administer three injections of 1 cc of 1% xylocalne (without epinephrine) to each of the anterolateral aspect of the distal thigh, and the anteromedial aspect of the proximal leg. Excellent anesthesia was observed, after which time a needle and syringe was used to administer 30 cc of bupivacaine to the intra-articular portion of the knee. The resulting anesthesia did not require any additional injections of anesthetic and the surgery proceeded without any problems.

For each study none of the patients experienced any fear of the needle-free injection system, in comparison to a needle and syringe. Additionally, the patients did not experience pain associated with the needle-free injection, and only experienced pain associated with the intra-articular bupivacaine injections.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

Inventions embodied in various combinations and subcombinations of features, functions, elements, and/or properties may be claimed through presentation of new claims in a related application. Such new claims, whether they are directed to a different invention or directed to the same invention, whether different, broader, narrower or equal in scope to the original claims, are also regarded as included within the subject matter of the inventions of the present disclosure.

What is claimed is:

1. A method of delivering at least one injectable to a joint region, comprising:
providing a needle-free injection system including a chamber for storing fluid and having a first end and a second end; an orifice of a pre-selected size in the first end of the chamber; and a plunger mechanism for ejecting a selectable amount of fluid from within the chamber, through the orifice, and onto a target site spaced at a selected interval from the orifice; wherein the chamber is at least partially filled with a first injectable; and the plunger mechanism is configured to eject a selected amount of the first injectable through the orifice and onto the target site at a pre-determined rate;
selecting a skin site adjacent a joint;
placing the orifice in an injection position relative to the skin site, whereby the skin site is the target site; and
ejecting the selected amount of the first injectable onto the skin site with the plunger mechanism, whereby at least a portion of the first injectable is delivered through the skin site to at least one of an intradermal, a subcutaneous, an intramuscular, or an intra-articular region of the joint.

2. The method of claim 1, wherein the first injectable includes compounds used in the prevention, diagnosis, alleviation, treatment, or cure of joint maladies, and wherein the compounds are selected from the group consisting of drugs, steroids, gels, anesthetics, antibiotics, and analgesics.

3. The method of claim 1, wherein the orifice has a diameter of about 0.0045 inches.

4. The method of claim 1, wherein the orifice has a diameter of about 0.015 inches.

5. The method of claim 1, wherein the needle-free injection system includes an adaptor having a proximal end selectively attachable to the first end of the chamber, and a distal end that is configured to engage a surface adjacent to a target site, wherein engaging the distal end of the adaptor with a skin surface adjacent to the skin site causes the orifice to be spaced at the selected interval from the skin site.

6. The method of claim 1, wherein the first injectable is only delivered to the intradermal region of the joint with the needle-free injection system.

7. The method of claim 1, wherein portions of the first injectable are delivered to each of the intradermal, the subcutaneous, and the intramuscular regions of the joint with the needle-free injection system.

8. The method of claim 1, wherein the skin site is adjacent a knee joint.

9. The method of claim 1, wherein the skin site is adjacent a hip joint.

10. The method of claim 1, wherein the skin site is adjacent an elbow joint.

11. The method of claim 1, wherein the skin site is adjacent a shoulder joint.

12. The method of claim 1, wherein the skin site is adjacent a vertebral joint.

13. The method of claim 1, wherein the first injectable includes an anesthetic that is delivered to at least one of the intradermal, the subcutaneous, or the intramuscular region of the joint with the needle-free injection system, and wherein the method further comprises delivering a selected amount of a second injectable to at least one of the intradermal, the subcutaneous, the intramuscular, or the intra-articular region of the joint with another injection system that includes a needle and syringe.

14. The method of claim 13, wherein the second injectable includes an anesthetic, and wherein the method further comprises performing arthroscopic surgery on the joint.

15. A method of delivering an injectable to a joint region with a needle-free injection system, comprising:
selecting a skin site adjacent a joint;
attaching, adjacent an injection orifice of a needle-free injection system, the proximal end of an adaptor having a proximal and distal end, to provide a fixed spacing from the injection orifice to the distal end of the adapter;
engaging the distal end of the adaptor with the skin site; and
using the needle-free injection system to inject an injectable through the skin site to at least one of an intradermal, a subcutaneous, an intramuscular, or an intra-articular region of the joint.

16. The method of claim 15, wherein the injection orifice is spaced at a distance of 1.5 inches from the distal end of the adapter.

17. A method of delivering an injectable to a joint with a needle-free injection system, comprising:
selecting a target joint of a subject;
providing a needle-free injection system having an injectable therein; and
effecting a release of the injectable whereby the injectable is accelerated to exit the needle-free injection system with sufficient energy as to penetrate into a target tissue proximal to the target joint.

18. The method of claim 19, wherein the injectable includes compounds used in the prevention, diagnosis, alleviation, treatment, or cure of joint maladies, and wherein the compounds are selected from the group consisting of drugs, steroids, gels, anesthetics, antibiotics, and analgesics.

19. The method of claim 18, wherein the target joint is selected from the group consisting of a hip, a knee, an ankle, a toe, a shoulder, an elbow, a wrist, a finger, and a vertebrae.
